(19) United States
(12) Patent Application Publication
(10) Pub. No.: US 2018/0303811 A1
(43) Pub. Date: Oct. 25, 2018

(54) ORAL SOLUTION OF DIHYDROPYRIDINE DERIVATIVES

(71) Applicant: FTF Pharma Private Limited, Taluka: Sanand Dist.: Ahmedabad (IN)

(72) Inventors: Jayanta Kumar Mandal, Satelite, Ahmedabad (IN); P. KANTEEPAN, Tamil Nadu (IN); Jay Rajesh BHAVSAR, Anand Nagar, Rajkot, Gujarat (IN); Dhaval VYAS, Thaltaje Ahmedabad Gujarat (IN)

(73) Assignee: FTF Pharma Private Limited, Taluka: Sanand Dist.: Ahmedabad (IN)

(21) Appl. No.: 15/769,406

(22) PCT Filed: Oct. 21, 2016
(86) PCT No.: PCT/IB2016/056327

§ 371 Date: Apr. 19, 2018

(30) Foreign Application Priority Data
Oct. 23, 2015 (IN) 3999/MUM/2015

(51) Int. Cl. A61K 31/4422; A61K 9/08; A61K 47/10; A61K 47/26

(52) U.S. Cl. CPC A61K 31/4422; A61K 47/26; A61K 47/10; A61K 9/08

(57) ABSTRACT

The present invention pertains to the field of oral pharmaceutical composition of dihydropyridine derivatives. In particular, present invention relates to oral solution formulations of dihydropyridines with non-aqueous solvent and antioxidant having improved stability.

### FIELD OF INVENTION

The present invention pertains to the field of oral pharmaceutical composition of dihydropyridine derivatives. In particular, present invention relates to oral solution formulations of dihydropyridines with non-aqueous solvent and antioxidant having improved stability.

### BACKGROUND OF INVENTION

Since their introduction in therapy more than 40 years ago, dihydropyridines have been amongst the most successful drugs ever used in humans. Dihydropyridines derivatives used in medical therapy are highly acclaimed as valuable and effective in treatment of cardiac muscle and circulation diseases.

The structure of dihydropyridine derivatives are based on a system of dihydropyridine with a nitro or chlorophenyl substituent at position 4, which makes these compound very sensitive to light and UV radiation. Further, dihydropyridine do not show any thermal degradation in dry air atmosphere in solid state but at elevated temperature in humid atmosphere these compounds undergo nitrozoderivative formation because of aromatisation of dihydropyridine ring by water molecule elimination.

Pharmaceutically acceptable acid Addition salt forms of dihydropyridine derivatives are disclosed in U.S. Pat. No. 4,572,909 which includes hydrochloride, hydrobromide, sulphate, phosphate or acid phosphate, acetate, maleate, fumarate, lactate, tartrate, citrate and gluconate salts of dihydropyridine derivatives.

Besylate salt of amlodipine is disclosed in U.S. Pat. No. 4,879,303. Further this patent discloses pharmaceutical dosage forms of the besylate salt of amlodipine such as tablet, capsule and sterile aqueous solution for parenteral administration.

WO2010070705/US2011294860 disclose an aqueous oral preparation of stable amlodipine with anionic surfactant having a sulfuric acid group or a sulfonic acid group which is either a liquid preparation or a jelly preparation having a pH of 4.5-7. Higher acidity of these formulations, because of presence of sulfuric acid group or a sulfonic acid group makes these formulations unfavorable from the perspective of children or aged patients.

To overcome above mentioned problem, present invention provides a patient friendly stable oral solution of dihydropyridines with antioxidant having improved thermal, light or UV stability over other oral dosage forms with improved palatability.

### OBJECT OF INVENTION

The primary object of present invention is to provide oral pharmaceutical solution of dihydropyridine derivatives with non-aqueous solvent and antioxidant having improved stability.

Another object of present invention is to provide a cost effective and highly patient compliance oral solution of derivatives of dihydropyridines.

Still another object of present invention is to provide process for preparation of oral pharmaceutical solution of dihydropyridine derivatives with non-aqueous solvent and antioxidant having improved stability.

### SUMMARY OF INVENTION

The present invention relates to an oral solution of dihydropyridine compounds with improved stability.

In accordance with one aspect of the present invention, there is provided an oral pharmaceutical solution of dihydropyridines with an antioxidant which stabilize derivatives of dihydropyridines from thermal degradation, light or UV radiation into solution form.

Another aspect of present invention relates to oral solution of dihydropyridine derivatives comprises of an active ingredient with an antioxidant and other pharmaceutically acceptable excipients such as vehicle, solvent, sweetener and flavouring agent.

### DETAIL DESCRIPTION OF INVENTION

Calcium channel blockers (CCBs) have a common mechanism of action, but there is difference in the subclasses based on pharmacological action they exert.

Dihydropyridine CCBs tends to be more potent vasodilators than non-dihydropyridine (non-DHP) agents, whereas the latter have more marked negative inotropic effects.

Dihydropyridine works via dual mode of action as following:
1) Inhibition of L-type calcium current which decreases intracellular calcium concentration and causes smooth muscle relaxation
2) Induces the release of nitric oxide (NO) from the vascular endothelium of various vessels and that provides beneficial antihypertensive effects of the dihydropyridine calcium antagonists.

Further, no release from both vascular endothelium and platelets may contribute to the antiatherosclerotic and antithrombotic effects of dihydropyridines.

Dihydropyridine (DHP) calcium channel blockers are derived from the molecule dihydropyridine and used to reduce systemic vascular resistance and arterial pressure as they have higher vascular selectivity and also used for treatment of angina and are particularly effecting for vasospastic angina as dihydropyridines have minimal effect on cardiac conduction or heart rate but have potent actions as arterial vasodilators.

The first-generation dihydropyridines have proven efficacy against hypertension, but they are associated with short duration and rapid onset of vasodilator action and more likely to be associated with adverse effects. In response of this problem second-generation dihydropyridines were synthesized which allowed better control of the therapeutic effect and have better pharmacokinetic profile that encompasses longer action than first-generation drugs. Second-generation dihydropyridines have enhanced vascular selectivity and have reduction in some adverse effects.

The third generation drugs have defined as long acting drugs and two distinct types of CCBs belong to this generation. One is characterized by a sustained blood concentration with a long half-life which is exemplified by amlodipine a one of the third generation dihydropyridine and pharmacodynamic innovation began with the third-generation agents including amlodipine, nitrendipine. The other category is characterized by lipophilic and highly histotropic properties which subsequently provides long-acting pharmacokinetics of this agents such as lercanidipine and lacidipine.

In an embodiment, the active pharmaceutical ingredient for oral pharmaceutical solution dosage form is selected from the derivatives of dihydropyridines such as nifedipine, nicardipine, felodipine, amlodipine, nitrendipine etc.

In an embodiment, the present invention is exemplified with one of the widely used and undeniably succeeded dihydropyridine derivative, amlodipine.

Chemically, amlodipine is 3-O-ethyl 5-O-methyl 2-(2-aminoethoxymethyl)-4-(2-chlorophenyl)-6-methyl-1,4-dihydropyridine-3,5-dicarboxylate. Amlodipine has following chemical structure:

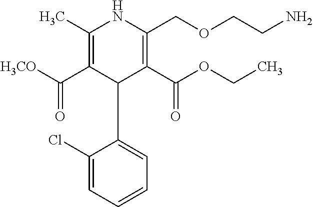 [SMILES: CCOC(=O)C1=C(COCCN)NC(C)=C(C(=O)OC)C1c1ccccc1Cl]

Salt is a product generated upon the neutralization of an acid or base. Pharmaceutical salts are important in the process of drug development, as converting an acidic or basic drug into a salt via a simple neutralization reaction has the ability to change the physicochemical properties of a drug.

Using different chemical species to neutralize the parent drug can produce a diverse series of compounds, and this process is traditionally used to improve drug stability, drug solubility and drug dissolution rates. In accordance with salt formation, various non-toxic Addition salts of amlodipine containing pharmaceutically acceptable anions such as hydrochloride, hydrobromide, sulfate, phosphate or acid phosphate, acetate, maleate, fumarate, lactate, tartrate, citronate and gluconate is synthesized to improve light and storage stability of amlodipine.

Besylate salt of amlodipine is synthesized for further improvement of stability. Sulfonic acid salts possess a range of properties that are useful to both synthetic and formulation chemist as they do offer significant advantages as alternatives to other salt forming moieties under certain circumstances. Besylate salt of amlodipine increases stability of amlodipine in liquid dosage form under various circumstances such as under refrigeration, room temperature conditions or thermal conditions.

In one of the embodiment of present invention, preferred salt form of amlodipine is amlodipine besylate and has following chemical structure:

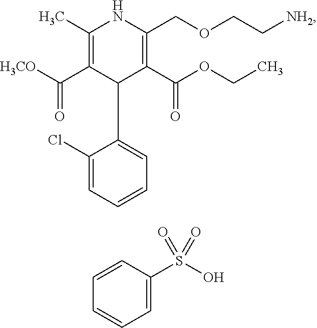

In one embodiment, present invention relates to an oral pharmaceutical solution of amlodipine besylate with improved stability in solution dosage form.

Aspects of present invention relates to oral pharmaceutical solution of amlodipine besylate comprising of active ingredient amlodipine besylate with antioxidant and other pharmaceutically acceptable excipients such as vehicle, solvent, sweetener, chelating agent, pH adjusting agent and flavouring agent.

Vehicles as referred to in the present invention, can be either aqueous vehicles or oily vehicles. Aqueous vehicles can be selected from but not limited to purified water, hydro-alcoholic, polyhydric alcohols and buffers, while oily vehicles can be selected from vegetable oils, mineral oils, organic oily bases or emulsified bases.

By solvents in the present invention, it refers to solvents which are used to increase solubility of drugs having low solubility in water. It is also used to improve viscosity, taste and flavor. Solvent system comprises of solvents selected from but not limited to propylene glycol, glycerin, alcohol, polyhydric alcohol and water for injection which is used alone or in combination.

The antioxidant and Non-aqueous solvent in the present invention, as long as it used to protect besylate salt of amlodipine from degradation into solution phase and not particularly limited. Antioxidant is a substance capable of inhibiting oxidation and that may be Added to pharmaceutical products to prevent deterioration by oxidative processes. Antioxidant can be selected from but not limited to butylated hydroxyanisole, butylated hydroxy toluene (BHT), sodium metabisulfite, ascorbic acid, alpha tocopherol, sodium edetate.

Sweetener referred in the present invention can be selected from but not limited to sucrose, sugar alcohol, liquid glucose, glycerol, sorbitol, saccharin sodium and aspartame to impart sweetness to the formulation.

By flavoring agents as referred to the present invention, are mainly use to increase the palatability and enhance the aesthetic qualities of the liquid formulation. Flavoring agent can be selected but not limited to oil based flavoring agent such as essential oils including peppermint oil, orange oil, and lemon oil etc or can be selected from fruit flavors.

### Example-1

The present invention can be described by way of example only. It is to be recognized that modifications falling within the scope and spirit of the description or claims, which would be obvious to a person skilled in the art based upon the disclosure herein, are also considered to be included within the scope of this disclosure.

For the composition of amlodipine besylate 1 mg/ml, drug and excipients with its range are shown below in table:

| | Drug/ | Quantity |
|---|---|---|
| No. | Excipients | (mg/ml) |
| 1 | Amlodipine besylate | 2.77 |
| 2 | Glycerin | 1000.0 |
| 3 | Liquid Maltitol | 140.0 |
| 4 | Butylated Hydroxy | 0.1 to 0.18 |
| | Toluene (BHT) | |
| 5 | Peach flavor | 0.5 |
| 6 | Purified water | Q.S. 1 ml |

The oral pharmaceutical solution of above composition is prepared by following method:—
a) Glycerine is heated to 80° C. and butylated hydroxy toluene Added to it and dissolved.
b) Amlodipine besylate is Added to the solution of step a) and dissolved into it.
c) Solution of step b) is allowed to cool to room temperature.
d) Liquid Maltitol is Added to solution of step c) and stirred.
e) Volume of solution of step d) is made up with purified water and stirred until homogeneous solution is obtained.
f) Solution of step e) is filled in 150 ml amber glass bottle secured with PP CR closure with (EPE wAdded).

### Example-2

For the composition of amlodipine besylate 1 mg/ml, drug and excipients with its range are shown below in table:

| | | Quantity |
|---|---|---|
| No. | Drug/Excipients | (mg/ml) |
| 1 | Amlodipine besylate | 2.77 |
| 2 | Glycerin | 1000.0 |
| 3 | Ethanol | 40 to 50 |
| 4 | Liquid Maltitol | 140.0 |
| 5 | Butylated Hydroxy | 0.1 to 0.18 |
| | Toluene (BHT) | |
| 6 | Peach flavor | 0.5 |
| 7 | Purified water | Q.S. 1 ml |

The oral pharmaceutical solution of above composition is prepared by following method:—
a) Glycerin is heated to 80° C. Amlodipine besylate is Added to the solution and dissolved into it. Cool the solution to room temperature
b) BHT was dissolved in ethanol and Added to above solution
c) Liquid Maltitol is Added to solution of step c) and stirred.
d) Volume of solution of step d) is made up with purified water and stirred until homogeneous solution is obtained.
e) Solution of step e) is filled in 150 ml amber glass bottle secured with PP CR closure with (EPE wAdded).

The oral pharmaceutical solution of above composition can also be prepared by using another method as under:—
a) Take glycerol 98% as total quantity of batch size
b) Take Ethanol absolute
c) Add Butylhydroxytoluene in Ethanol absolute
d) Add amlodipine besilate in step c the whitish dispersion obtaine
e) Add remaining 2% glycerol in step c to obtain clear pale straw color viscous solution
f) Transfer solution from (step e in to step a) with continuous stirring to obtain clear pale straw color viscous solution
g) Add liquid maltitol with continuous stiffing to obtain clear pale straw color viscous solution
h) Add Flavour with continuous stiffing to obtain clear pale straw color viscous solution
i) Add purified water to make up continuous stiffing to obtain clear pale straw color viscous solution
j) Filter the solution 40 μm PP filter. Fill in the 150 ml USP-III amber glass bottle with CRC

Using the above composition of example 1 and 2, suspension can also be prepared by following method:—
a) Take 70% Purified water of total batch size, heated to 80-85° and Glycerin. Allow to cool at room temperature.
b) Add and dissolve liquid maltitol or in step 1.0 under stiffing.
c) Add and dissolve API (active pharmaceutical ingredient) in step 2.0 under stiffing.
d) Butylated Hydroxy Toluene (BHT)
e) Add and mixed flavouring agent in step 5.0 under stirring.
f) Finally volume makes up by Purified water and mix properly under stirring.

We claim:

1) A pharmaceutical oral solution with improved stability of dihydropyridine derivative with major amount of non-aqueous solvent and antioxidant.

2) A pharmaceutical oral solution of dihydropyridine derivative as claimed in claim 1, wherein dihydropyridine derivative can be selected from nifedipine, nicardipine, felodipine, amlodipine, nitrendipine or a pharmaceutically acceptable salt thereof.

3) A pharmaceutical oral solution of dihydropyridine derivative as claimed in claim 2, wherein dihydropyridine derivative is amlodipine or a pharmaceutically acceptable salt thereof.

4) A pharmaceutical oral solution of dihydropyridine derivative as claimed in claim 1, wherein other pharmaceutically accepted excipients are selected from solvent, vehicle, sweetener and flavoring agent.

5) A process for preparation of a pharmaceutical oral solution with improved stability of amlodipine besylate comprises of following steps:
a) Heating of glycerine at 80° C. and dissolution of butylated hydroxy toluene into it.
b) Addition and dissolution of amlodipine besylate to solution of step a).
c) Cooling of solution of step b) to room temperature.
d) Addition of liquid maltitol to solution of step c) and stir it.
e) Make up the volume of step d) with purified water and stir it until homogeneous solution is obtained.

6) A process for preparation of a pharmaceutical oral solution with improved stability of amlodipine besylate comprises of following steps:
a) Take glycerol 98% as total quantity of batch size
b) Take Ethanol absolute
c) Add Butylhydroxytoluene in Ethanol absolute
d) Add amlodipine besilate in step c the whitish dispersion obtaine
e) Add remaining 2% glycerol in step c to obtain clear pale straw color viscous solution
f) Transfer solution from (step e in to step a) with continuous stirring to obtain clear pale straw color viscous solution
g) Add liquid maltitol with continuous stiffing to obtain clear pale straw color viscous solution
h) Add flavouring agent with continuous stiffing to obtain clear pale straw color viscous solution
i) Add purified water to make up continuous stiffing to obtain clear pale straw color viscous solution
j) Filter the solution 40 μm PP filter. Fill in the 150 ml USP-III amber glass bottle with CRC

7) A pharmaceutical oral suspension with improved stability of dihydropyridine derivative with major amount of non-aqueous solvent and antioxidant.

8) A pharmaceutical oral suspension with improved stability of dihydropyridine derivative as claimed in claim 7, wherein suspension is prepared by following method:—
a) Take 70% Purified water of total batch size, heated to 80-85° and Glycerin. Allow to cool at room temperature.
b) Add and dissolve liquid maltitol or in step 1.0 under stiffing.
c) Add and dissolve API (active pharmaceutical ingredient) in step 2.0 under stiffing.
d) Butylated Hydroxy Toluene (BHT)
e) Add and mixed flavouring agent in step 5.0 under stiffing.
f) Finally volume makes up by Purified water and mix properly under stirring.
g) Filling of Solution of step e) in 150 ml amber glass bottle secured with PPCR closure with (EPE wAdded).
